(19) United States
(12) Patent Application Publication
(10) Pub. No.: US 2010/0234876 A1
(43) Pub. Date: Sep. 16, 2010

(54) APPARATUS AND METHODS FOR RECAPTURING AN ABLATION BALLOON

(75) Inventor: James R. Watson, Santa Rosa, CA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 12/719,582

(22) Filed: Mar. 8, 2010

Related U.S. Application Data
(60) Provisional application No. 61/158,917, filed on Mar. 10, 2009.

(51) Int. Cl. A61M 29/02

(52) U.S. Cl. 606/194

(57) ABSTRACT

A recapturing apparatus, a medical kit including the recapturing apparatus and a method for performing a medical procedure using the recapturing apparatus are provided. The recapturing apparatus includes a moveable collar and a push wire. The moveable collar comprises a proximal attachment region and a distal capture region sized to capture an expandable body therein. The push wire is configured to couple to the attachment region. The medical kit includes a sheath, a catheter having a distally-located expandable body, and the recapturing apparatus located concentrically between the sheath and the catheter. The method for performing the medical procedure includes coupling the push wire to the collar and advancing the collar distally along the catheter until the expandable body on the catheter is captured within the collar.

### RELATED APPLICATION DATA

The present application claims the benefit under 35 U.S.C. §119 to U.S. provisional patent application Ser. No. 61/158,917, filed Mar. 10, 2009. The foregoing application is hereby incorporated by reference into the present application in its entirety.

### FIELD OF THE INVENTION

The disclosed inventions relate to apparatus and methods for performing a medical procedure with a catheter balloon, and, more particularly, to safely removing the catheter balloon from a patient's body after the medical procedure.

### BACKGROUND

Atrial fibrillation is a condition in which upper chambers of the heart beat rapidly and irregularly. One known manner of treating atrial fibrillation is to administer drugs in order to maintain normal sinus rhythm and/or to decrease ventricular rhythm. Known drug treatments, however, may not be sufficiently effective, and additional measures such as cardiac tissue ablation must often be taken to control the arrhythmia. Known ablation procedures for treating atrial fibrillation include performing transmural ablation of the heart wall or adjacent tissue walls using radio frequency (RF) energy. One known ablation procedure involves burning or ablating cardiac tissue and forming lesions to break up circuits believed to drive atrial fibrillation.

In addition to RF transmural ablation, cryogenic ablation has received increased attention for treatment of atrial fibrillation in view of the effectiveness of cryo-ablation procedures with fewer side effects. One known endocardial cryo-ablation procedure involves inserting a catheter into the heart, e.g., through the leg of a patient. Once properly positioned, a portion of the catheter, typically the tip of the catheter, is cooled to a sufficiently low temperature by use of a liquid coolant or refrigerant such as nitrous oxide, e.g., to sub-zero temperatures of about −75° C., in order to freeze tissue believed to conduct signals that cause atrial fibrillation. The frozen tissue eventually dies so that the ablated tissue no longer conducts electrical impulses that are believed to cause or conduct atrial fibrillation signals.

Certain known endocardial cryo-ablation devices include expandable balloons, which are inflated with the liquid coolant or refrigerant. After the ablation is performed and before the device is withdrawn from the patient, the balloon is deflated and retracted into a guide sheath. During the endocardial cryo-ablation procedure, the catheter comprising a cryo-ablation balloon at the distal end thereof may be introduced into the left atrium of the heart using a conventional retrograde approach, i.e., through the respective aortic and mitral valves of the heart. Alternatively, a more simple approach is to introduce the catheter from the right atrium into the left atrium using a transeptal approach through the fossi ovalis. A detailed description of the transeptal approach is disclosed in U.S. Pat. No. 5,575,810, issued to Swanson et al., which is fully and expressly incorporated herein by reference.

However, after expanding the balloon, performing the ablation procedure, and deflating the balloon, a user may encounter difficulties in retracting the deflated balloon from the left atrium back through the atrial septum into the right atrium. In particular, prior to inflation, the balloon profile is at its smallest, but after inflation has occurred, the balloon material is free to expand and may bunch up at the atrial septum during retraction of the deflated balloon back through the atrial septum. Thus, increased force is required to retract the deflated balloon, thereby potentially damaging the atrial septum during the balloon retraction procedure.

### SUMMARY OF THE INVENTION

In accordance with a first aspect of the disclosed inventions, a recapturing apparatus is provided. The recapturing apparatus comprises a moveable collar sized to fit within a guide sheath and comprises a central lumen sized to slidably receive a catheter shaft therein. The collar has a proximal attachment region and a distal capture region sized to snugly fit over and radially compress an expandable body disposed at a distal end of the catheter shaft. In one embodiment, an inner diameter of a distal end of the collar is greater than an inner diameter of a proximal end of the collar. The recapturing apparatus further comprises a push wire, the distal end of which is configured to engage the attachment region of the collar, thereby affixing the push wire to the collar. The distal end of the push wire may be interference fit with the attachment region of the collar.

By way of non-limiting example, the attachment region of the collar may have an opening for receiving the distal end of the push wire therein. In this case, the distal end of the push wire may comprise flared ridges configured for engaging the opening in the attachment region of the collar. The opening in the attachment region of the collar may be substantially parallel to the central lumen in the collar, in which case, the distal end of the push wire is straight, or the opening may be perpendicular to the central lumen in the collar, in which case, the distal end of the push wire may be bent at a substantially right angle relative to a proximal portion of the push wire.

As another example of an interference fit arrangement, the attachment region of the collar comprises a pin and the distal end of the push wire comprises an opening for receiving the pin therein. In this case, the opening in the distal end of the push wire may be configured to engage with the pin in the attachment region of the collar by receiving the pin within the opening and then rotating the push wire from a perpendicular position to a parallel position relative to the lumen of the collar. In another embodiment, the attachment region further comprises an opening adjacent to the pin. In this case, the opening in the distal end of the push wire may be configured to engage with the pin in the attachment region of the collar by inserting the distal end of the push wire into the opening in the attachment region of the collar and then rotating the push wire from a perpendicular position to a parallel position relative to the lumen of the collar. The distal end of the push wire may have a quadrate cross-section and a proximal portion of the push wire may have a rounded cross-section.

In accordance with another aspect of the disclosed inventions, a medical kit is provided, the medical kit including an elongated catheter having a distally located expandable body (e.g., a balloon) configured for being placed between an expanded geometry and a collapsed geometry. In one embodiment, the expandable body is configured for ablating tissue. The medical kit further comprises a moveable collar configured for being displaced relative to the catheter to capture the expandable body therein when in the collapsed geometry, and an elongated push wire configured for engaging the moveable collar. As examples, the above-mentioned means for attaching the push wire to the collar can be used. The moveable collar and push wire may include any of the detailed features, including the attachment means, discussed above. The medical kit optionally includes an elongated sheath, in which case, the catheter is configured for being slidably disposed within the lumen of the sheath, and the moveable collar is configured for being disposed concentrically between the catheter and the sheath.

In accordance with still another aspect of the disclosed inventions, a method for performing a medical procedure using a catheter having an expandable body (e.g., a balloon) is provided. The method includes advancing the catheter through a sheath until the expandable body exits the sheath, expanding the expandable body, performing the medical procedure (e.g., a tissue ablation procedure), collapsing the expandable body after performing the medical procedure, and advancing a collar along the catheter until the expandable body is captured within the collar. In one method, the collar is concentrically located between the catheter and the sheath. The method also comprises retracting the collar into the sheath. As one example, an elongated push wire may be coupled to the collar (e.g., by interference fitting the push wire to the collar), such that the collar can be advanced along the catheter by advancing the push wire relative to the catheter.

One exemplary method comprise moving the expandable body from a first anatomical cavity (e.g., a right atrium) into a second anatomical cavity (e.g., a left atrium) by passing through a septum (e.g., an atrial septum) between the respective cavities, wherein expanding the expandable body, performing the medical procedure, collapsing the expandable body, and advancing the collar are performed while the expandable body is in the second cavity. The expandable body may be passed through an opening within the septum, in which case, the expandable body, after the step of collapsing the expandable body, may have a greater profile than the opening. A distal end of the sheath may remain in the first cavity while the expandable body is moved from the first cavity into the second cavity, in which case, the collar and the captured expandable body may be withdrawn from the second cavity into the first cavity by passing through the septum prior to retracting the collar into the sheath.

### BRIEF DESCRIPTION OF THE DRAWINGS

Referring now to the drawings in which like reference numbers represent corresponding parts throughout and in which:

FIG. 1A is an exploded plan view of a medical kit assembly including a recapturing apparatus, constructed in accordance with the disclosed inventions;

FIG. 1B is a cross sectional view of a proximal end of the medical kit assembly prior to deployment of the recapturing apparatus;

FIG. 1C is a cross sectional view of the proximal end of the medical kit assembly during deployment of the recapturing apparatus;

FIG. 2A is a cross sectional view of the recapturing apparatus according to one embodiment;

FIG. 2B is a cross-sectional view of an alternative embodiment of the recapturing apparatus shown in FIG. 2A;

FIG. 3A is a top plan view of the recapturing apparatus according to another embodiment;

FIGS. 3B and 3C are cross sectional views of the recapturing apparatus taken along lines 3B-3B and 3C-3C, respectively, in FIG. 3A;

FIG. 3D is a cross sectional view of an alternative embodiment of the recapturing apparatus depicted in FIGS. 3A-3C;

FIG. 4A is a top plan view of another embodiment of a push wire;

FIGS. 4B-4D are cross sectional views of the push wire taken along lines 4B-4B, 4C-4C and 4D-4D, respectively, in FIG. 4A;

FIG. 4E is a top plan view of an alternative embodiment of the push wire depicted in FIGS. 4A-4D;

FIG. 5A is a cross sectional view of a recapturing apparatus according to still another embodiment;

FIG. 5B is a top plan view of the recapturing apparatus shown in FIG. 5A;

FIG. 6 is a cross sectional view of a recapturing apparatus according to still another embodiment;

FIG. 7A is a cross sectional view of a recapturing apparatus according to yet another embodiment;

FIG. 7B is a cross sectional view of an alternative embodiment of the recapturing apparatus shown in FIG. 7A; and

FIGS. 8A-8F are partial cross sectional views of steps in a method of using the medical kit shown in FIGS. 1A-1C for performing a medical procedure.

### DETAILED DESCRIPTION OF ILLUSTRATED EMBODIMENTS

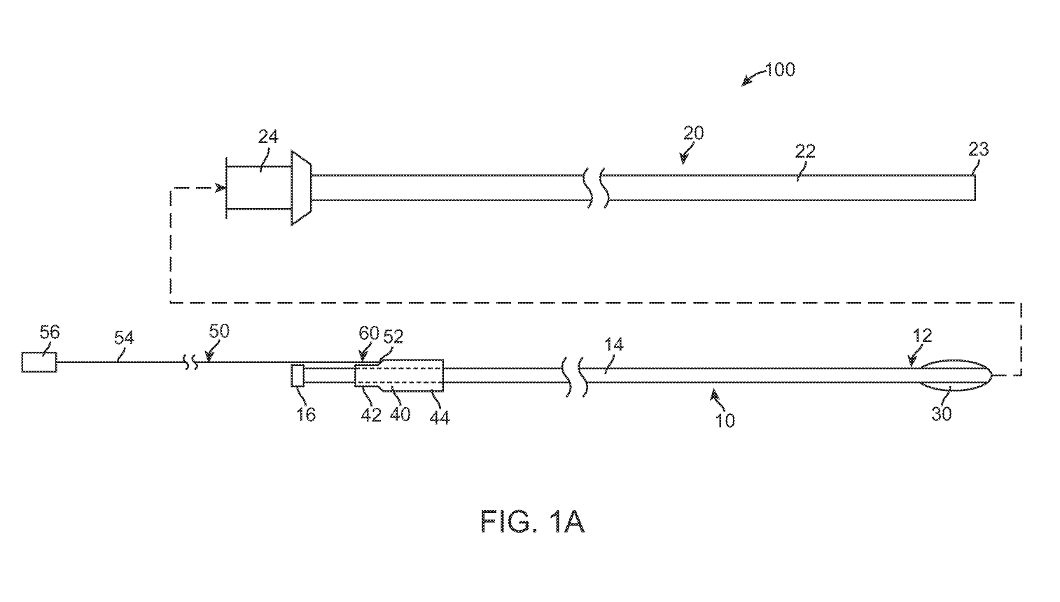

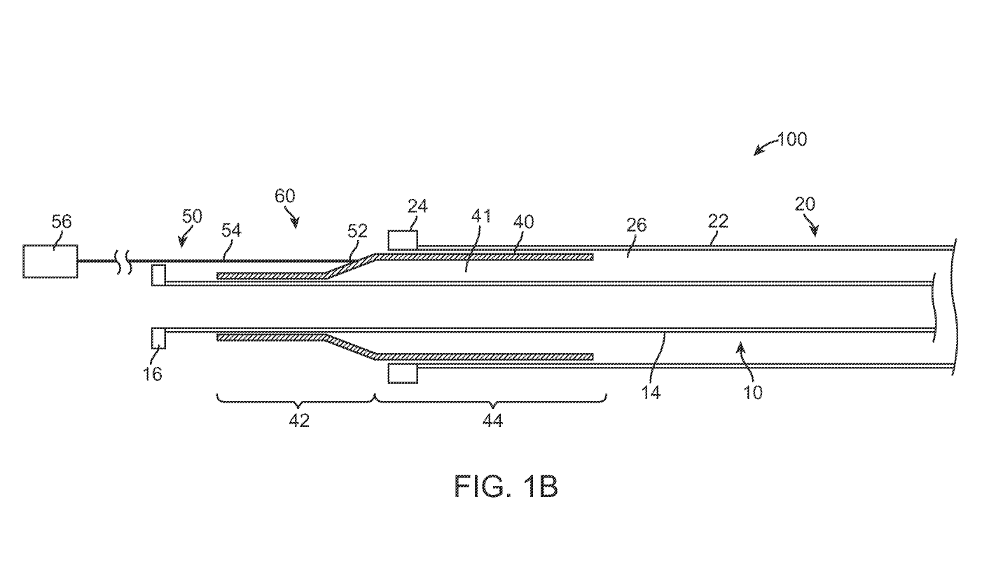

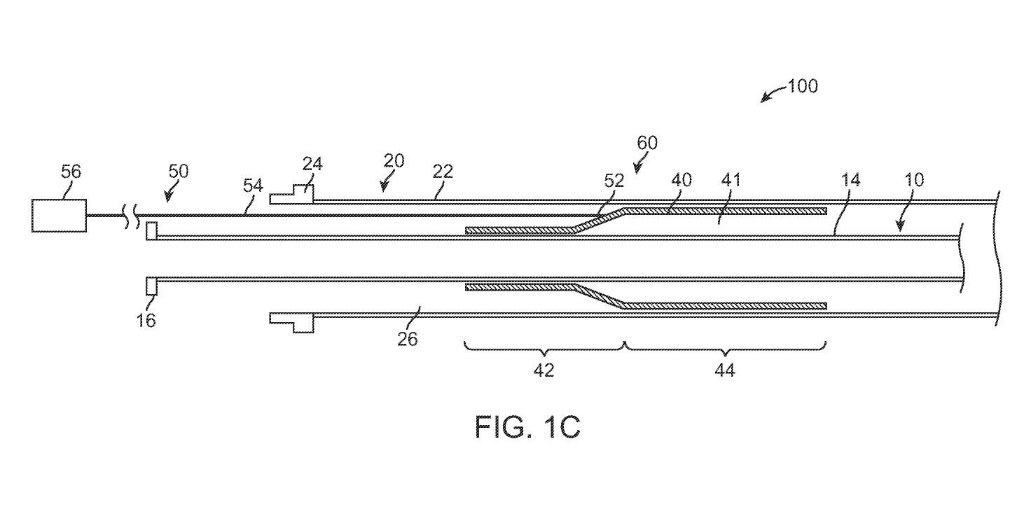

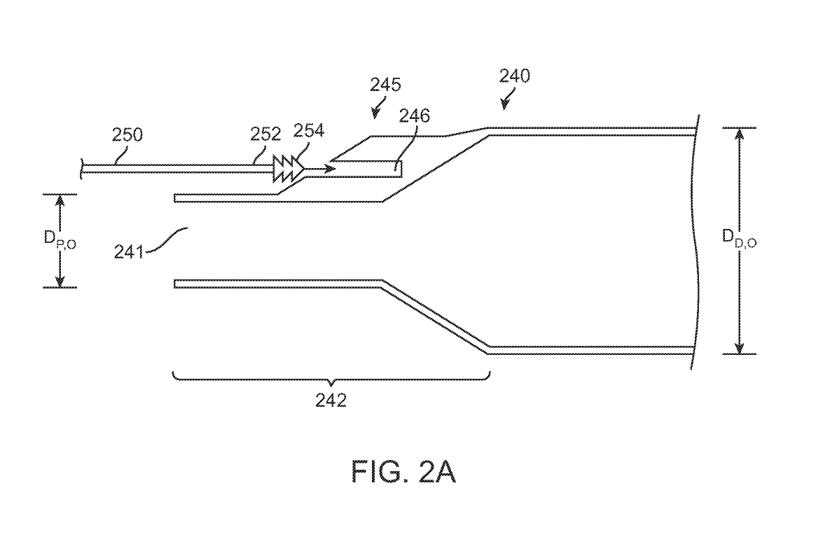

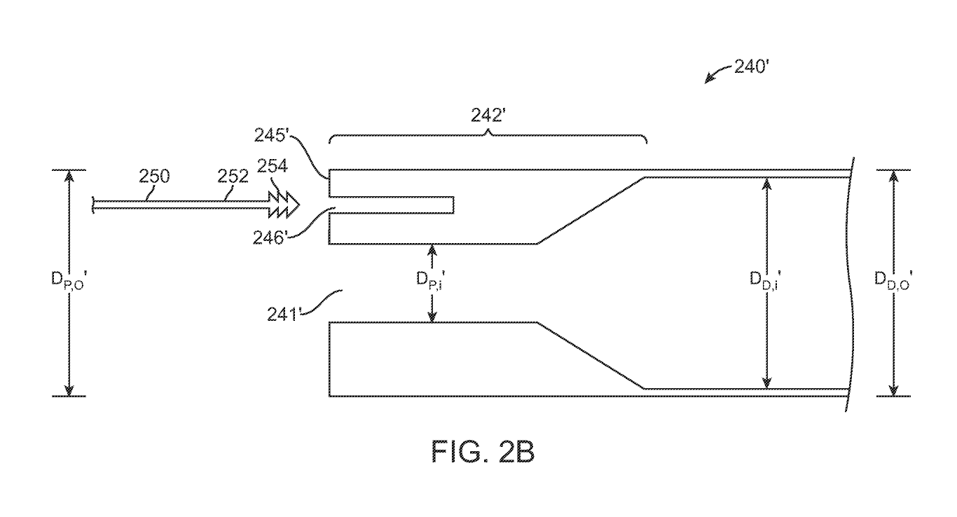

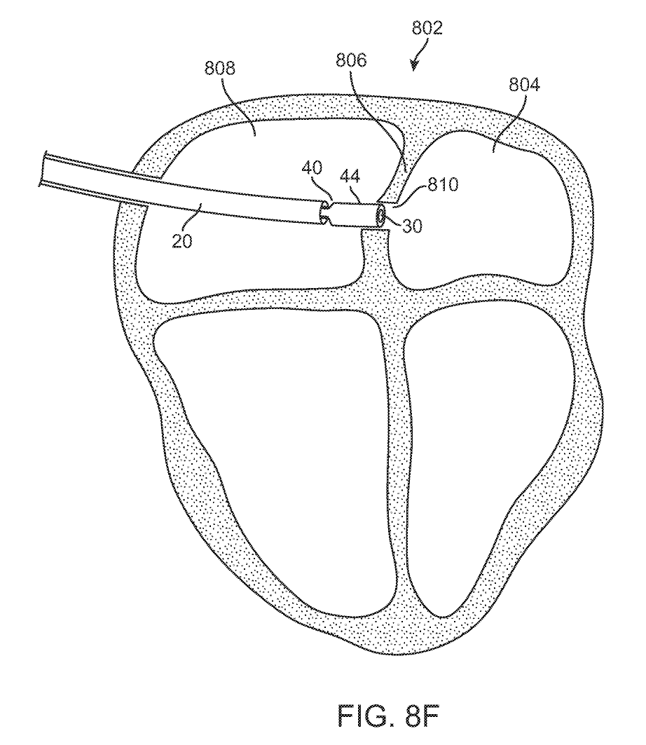

Embodiments relate to apparatus and methods for recapturing a deflated balloon, such that the balloon may easily be retracted into a sheath and safely removed from a patient's body. In this manner, embodiments of a recapturing apparatus advantageously include a moveable collar and a push wire for coupling to an attachment region of the collar and distally advancing the collar until the deflated balloon is captured within a capture region of the collar. With the deflated balloon captured within the moveable collar, retraction of the deflated balloon through an opening in an atrial septum is easier compared to known devices, which may encounter difficulty in retracting the deflated balloon through the atrial septum, as discussed above. Embodiments are described in further detail with reference to FIGS. 1A-1C, which illustrate a medical kit including a recapturing apparatus, FIGS. 2A-7B, which illustrate various embodiments for coupling the push wire to the moveable collar, and FIGS. 8A-8F, which illustrate steps in a method of using the medical kit.

Referring to FIGS. 1A-1C, an exemplary medical kit 100 constructed in accordance with the disclosed inventions is shown. The medical kit 100 generally includes a catheter 10, a guide sheath 20 sized for slidably receiving the catheter 10 therein, and a recapturing apparatus 60 sized for being disposed concentrically between the catheter 10 and the sheath 20. The medical kit 100 may further include a guide wire and/or other therapeutic tools (not shown).

The catheter 10 generally comprises a catheter body 14, a handle 16 mounted to the proximal end of the catheter body 14, and a balloon 30 mounted to the distal end 12 of the catheter body 14. In the illustrated embodiment, the catheter 10 is an ablation catheter and the balloon 30 is a cryo-ablation balloon.

The guide sheath 20 generally comprises an elongated body 22, a handle 24 coupled to a proximal end of the elongated body 22, and a lumen 26 extending through the elongated body 22 for allowing the catheter 10, guide wire (not shown), and/or other therapeutic tools (also not shown) to be inserted from the proximal end of the handle 24 towards the distal end 23 of the elongated body 22. The distal end 23 of the elongated body 22 is configured to be introduced through the vasculature of the patient, and into an anatomical cavity, such as the left atrium of the heart.

The recapturing apparatus 60 generally comprises a moveable collar 40 and a push wire 50 configured for being coupled to the collar 40. The push wire 50 comprises an elongated body 54 and a handle 56 on the proximal end of the elongated body 54. The moveable collar 40 includes a central lumen 41 for slidably receiving the catheter 10 therein, and an outer diameter sized for being slidably received within the guide sheath 20. The movable collar 40 also includes a proximal attachment region 42 configured for engaging with the distal end 52 of the push wire 50, and a distal capture region 44 sized to snugly fit over and radially compress the balloon 30, when deflated. To this end, the distal end of the collar 40 has an inner diameter large enough to facilitate recapturing the balloon 30, while the proximal end has an inner diameter large enough to pass the catheter body 14 therethrough, but small enough to accommodate a means for attaching the push wire 50 to the collar 40, as discussed in greater detail below. Thus, the inner diameter of the distal end of the collar 40 is larger than that of a proximal end of the collar 40. The disclosed inventions contemplate the use of several different embodiments of the attachment means for coupling the distal end 52 of the push wire 50 to the attachment region 42 of the collar 40.

In one mechanism for coupling the push wire to the collar, the distal end of the push wire is received into an opening in the attachment region of the collar and held in place by friction or interference fit. For example, in one embodiment shown in FIG. 2A, an attachment region 242 of a collar 240 includes an opening 246 for receiving the distal end 252 of a push wire 250 therein. The longitudinal axis of the opening 246 is substantially parallel to the longitudinal axis of the central lumen 241 in the collar 240. Also, the outer diameter of the distal end, D_{D,o}, is greater than the outer diameter of the proximal end, D_{P,o}, so that the distal end is large enough to capture the balloon 30, while the proximal end is sized to accommodate a mechanism on the outside of the collar 240 to which the push wire 250 can attach. In this case, the mechanism on the outside of the collar 240 to which the push wire 250 can attach is a ramped portion 245 of the collar 240 in which the opening 246 is disposed.

The distal end 252 of the push wire 250 includes flared ridges 254 configured for an interference fit within the opening 246 in the attachment region 242 of the collar 240. It should be well understood that other interference fit configurations are possible. For example, the push wire distal end 252 may have an outer diameter equal to or slightly larger than the inner diameter of the opening 246, so that the push wire 250 is configured for being interference fit within the opening 246 in the collar 240.

In an alternative embodiment shown in FIG. 2B, a collar 240′, like the collar 240 described above, includes an attachment region 242′ having an opening 246′ for receiving the distal end 252 of the push wire 250 therein. However, the outer diameter diameters D_{D,o}′, D_{P,o}′ at the distal and proximal ends of the collar 240′ are equal, while the inner diameter D_{P,i}′ at the proximal end is smaller than the inner diameter D_{D,i}′ at the distal end (i.e., the proximal end of the collar 240′ has a wall thickness that is greater than that of the distal end of the collar 240′). This allows the wall thickness of the collar 240′ to serve as the attachment region 242′, with the opening 246′ being formed in the proximal facing surface 245′ of the attachment region 242′.

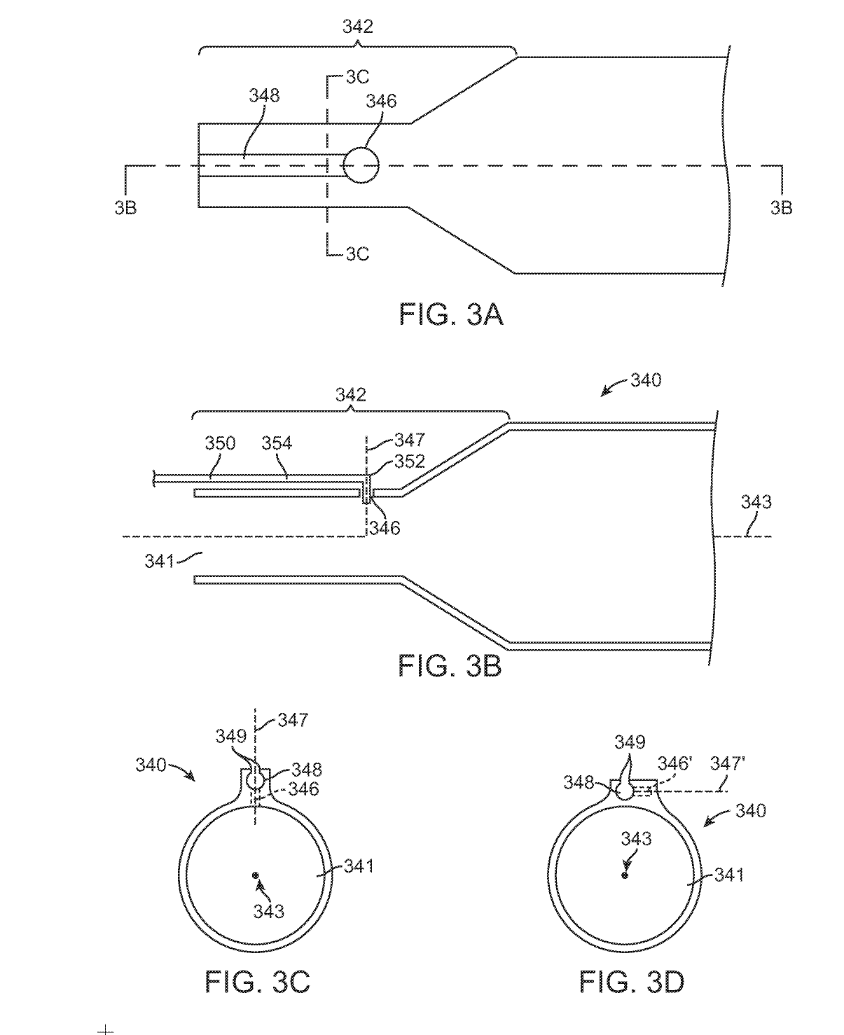

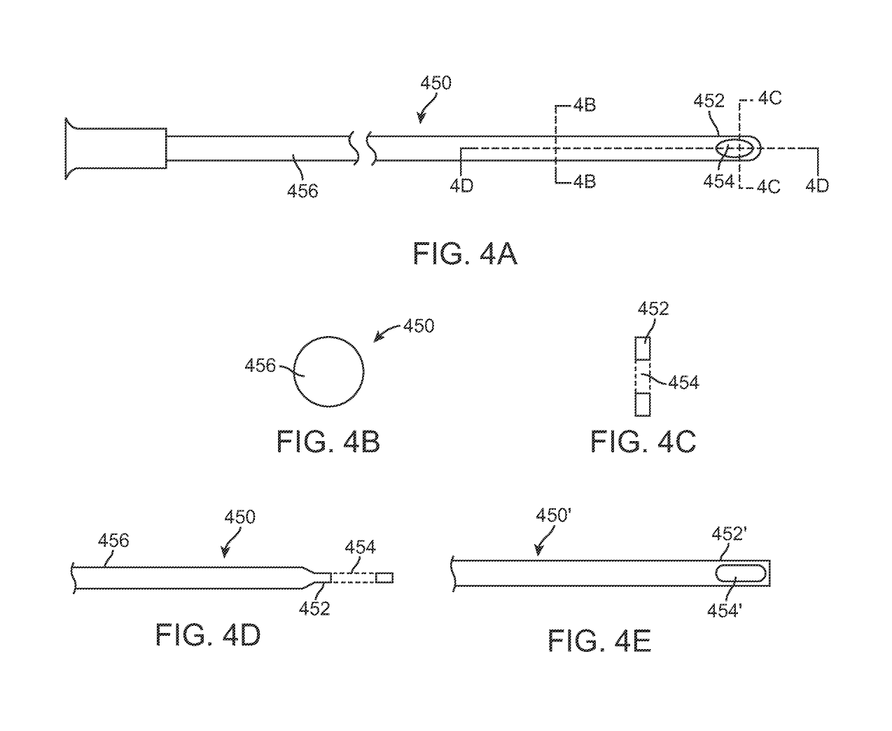

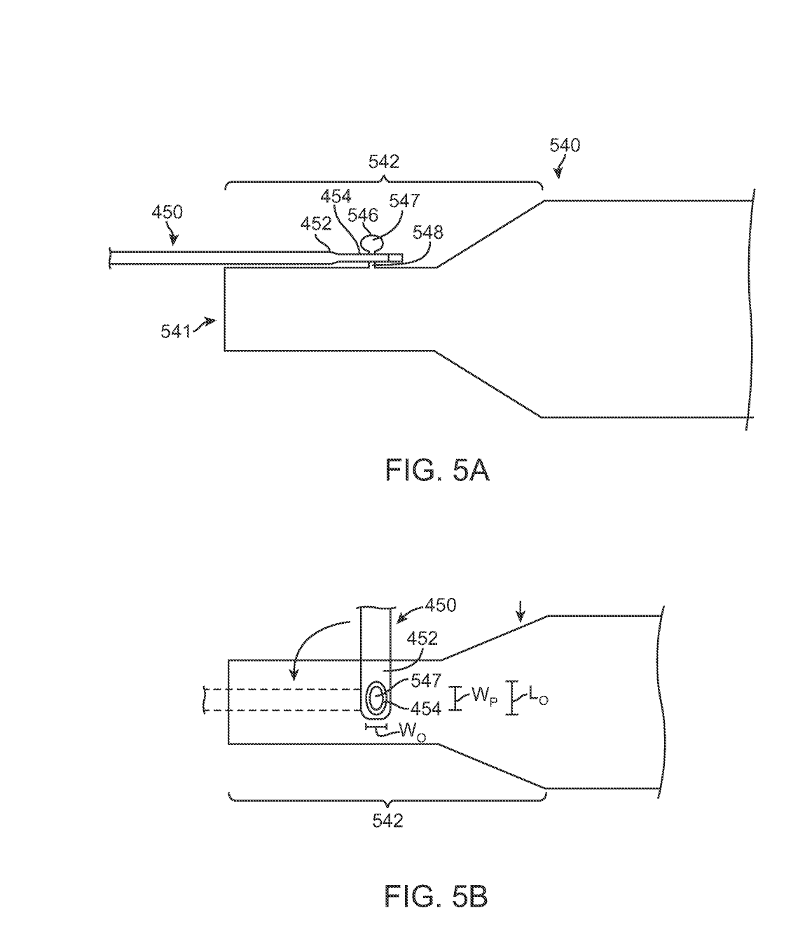

In the previously described attachment regions 242 and 242′, the longitudinal axes of the corresponding openings 246 and 246′ are parallel to the longitudinal axis of the central lumens 241 and 241′ of the collars 240 and 240′. However, openings within the attachment regions can be perpendicular to the longitudinal axis of the central lumen. For example, as shown in FIGS. 3A-3D, an attachment region 342 of a moveable collar 340 includes an opening 346 with a longitudinal axis 347 that is substantially perpendicular to the longitudinal axis 343 of the central lumen 341 in the collar 340. In this case, the distal end 352 of a push wire 350 is bent at a substantially right angle relative to a shaft portion 354 of the push wire 350.

In an exemplary embodiment, the attachment region 342 includes an external groove 348 parallel to the lumen 341 of the collar 340 for stabilizing the push wire 350 relative to the collar 340. The opening 346 is located at the distal end of the groove 348, such that the shaft 354 of the push wire 350 is held in place relative to the collar 340 when the bent distal end 352 is located within the opening 346. Such a groove 348 has a profile slightly larger than the circumference of the push wire 350. In the illustrated embodiment, the attachment region 342 has protruding clip portions 349 that oppose each other to form the groove 348. The material of the collar 340 is flexible enough to allow the clip portions 349 to open slightly so that the push wire 350 can be inserted between them, and is elastic enough to return to its original configuration after the push wire 350 passes between the clip portions 349, thereby retaining the push wire 350 within the groove 348. In an alternative embodiment, shown in FIG. 3D, the opening 346′ in the attachment region 342 is offset by ninety degrees relative to the opening 346 shown in FIGS. 3A-3C and also comprises a longitudinal axis 347′ that is perpendicular to the longitudinal axis 343 of the central lumen 341 in the collar 340.

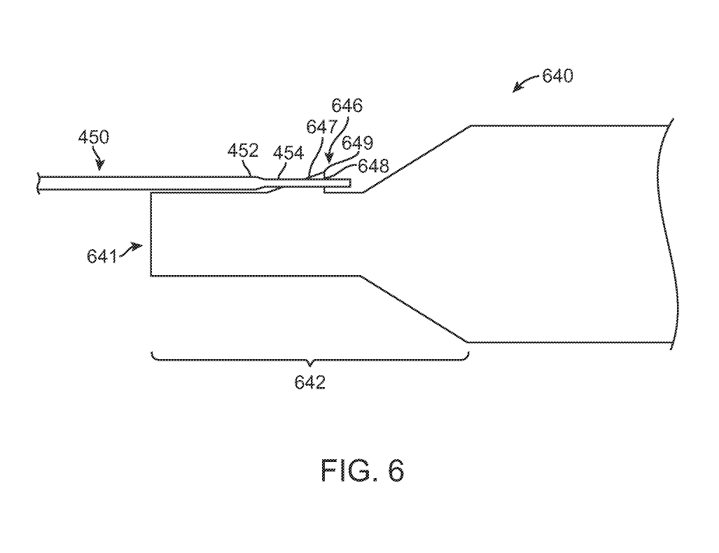

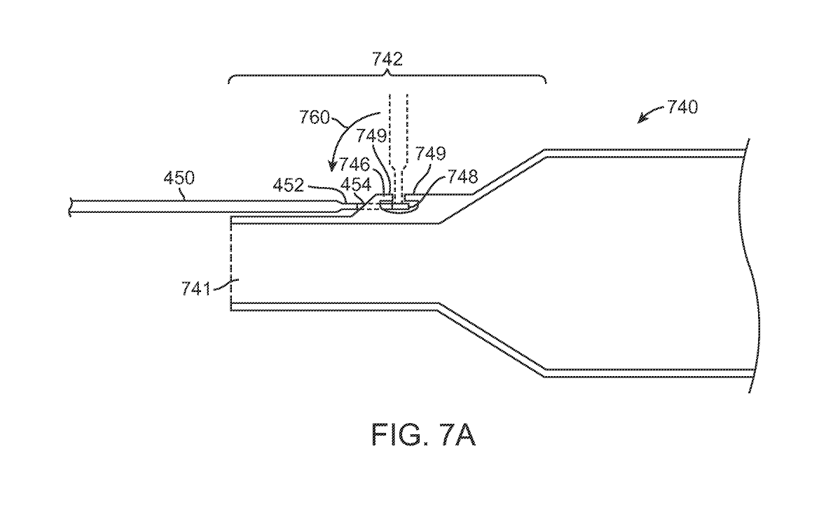

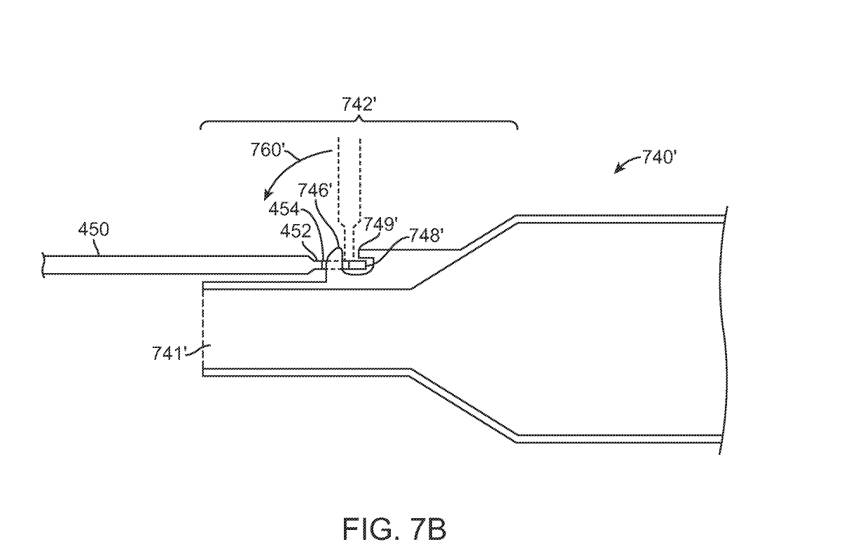

Although the previous embodiments of the attachment regions have openings for receiving the distal end of a push wire, attachment regions may, instead, include a protrusion or pin, while the distal end of the push wire includes an opening for receiving the pin therein. For example, FIGS. 4A-4D depict one embodiment of a push wire 450 with an opening 454 in the distal end 452 thereof configured for receiving the attachment region pin (discussed in more detail below) therein. The proximal portion 456 of the push wire 450 has a circular cross-section, as shown in FIG. 4B, and the distal end 452 of the push wire 450 has a quadrate cross-section, as shown in FIG. 4C. Further, the distal end 452 is flattened relative to the proximal portion 456, as best shown in FIG. 4D. Although FIG. 4A depicts the distal end 452 as being rounded, it should be well understood that the distal end 452 may have any desired shape. For example, in an alternative embodiment shown in FIG. 4E, the distal end 452′ has a rectangular shape. Similarly, although the opening 454 is shown as having an oval or elliptical shape, the opening 454 may alternatively have other shapes as desired.

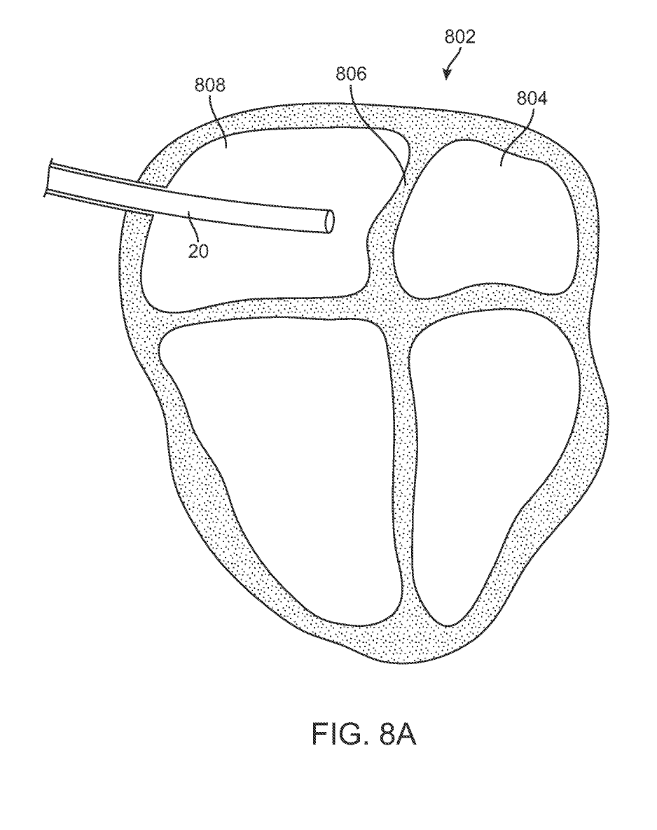

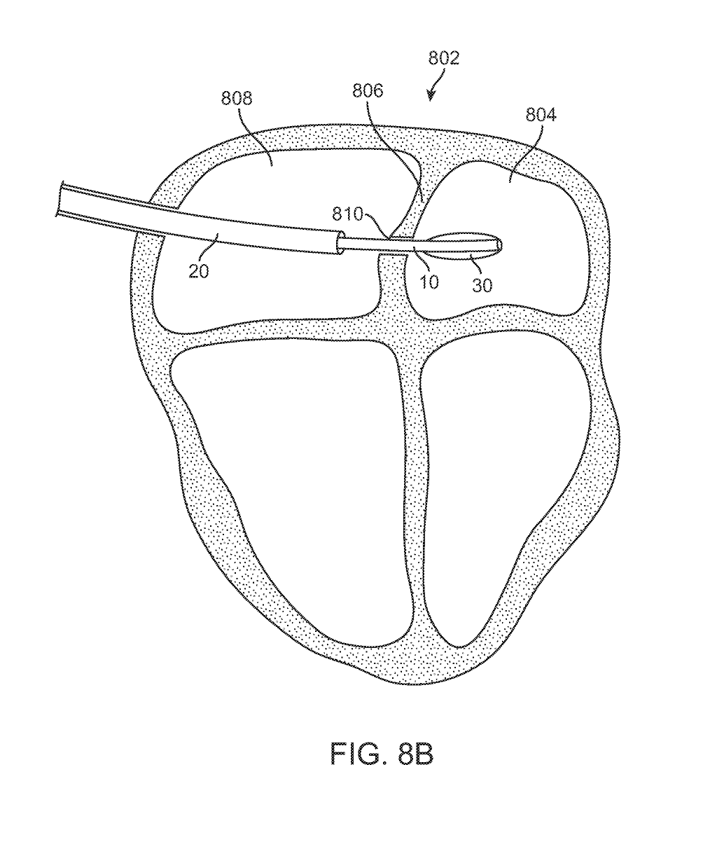

In another embodiment, the push wire depicted in FIGS. 4A-4E can be used with a collar comprising a pin with a smaller stem portion and a larger, bulbous top portion for retaining the push wire 450 in place relative to the collar as described in more detail below. For example, FIGS. 5A and 5B show one embodiment of a moveable collar 540 that is configured to couple to the push wire 450 depicted in FIGS. 4A-4E. An attachment region 542 of the collar 540 has a protruding pin 546 with an oval or elliptical-shaped bulbous top portion 547 and a thinner stem portion 548. The pin 546 is sized to fit within the opening 454 when the push wire 450 is in a perpendicular position relative to the collar lumen 541, as shown in FIG. 5B, and to be retained within the opening 454 when the push wire 450 is in a parallel position relative to the collar lumen 541, as shown in FIG. 5A. To this end, the bulbous top portion 547 of the pin 546 has a width, Wₚ, that is greater than the width, Wₒ, of the opening 454 in the push wire 450, but slightly smaller than the length, Lₒ, of the opening 454 in the push wire 450. Thus, the opening 454 is configured to pass over the bulbous top portion 547 of the pin 546 while the push wire 450 is in the perpendicular position shown in FIG. 5B, and then trapped by the bulbous top portion 547 while the push wire 450 is in the parallel position shown in FIG. 5A.

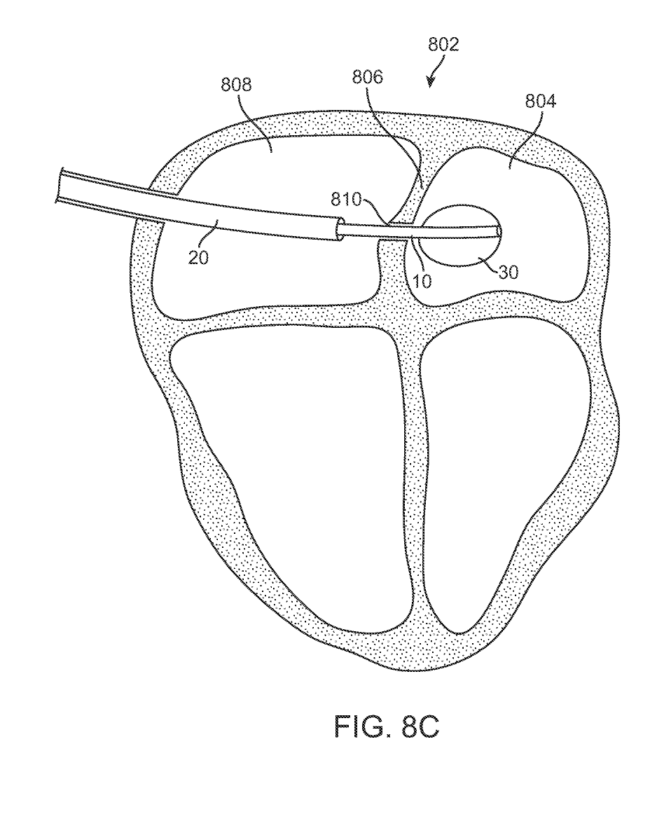

In still another embodiment, the push wire depicted in FIGS. 4A-4E can be used with a collar comprising a ramp-shaped protrusion or pin, where the apex of the ramp fits within the opening 454 in the distal end 452 of the push wire 450 without requiring rotation of the push wire 450. For example, FIG. 6 depicts a moveable collar 640 comprising an attachment region 642 having a ramp-shaped protrusion 646 with a proximal tapered surface 647, a distal blunt surface 648, and an apex 649 at the junction between the two surfaces 647 and 648. The apex 649 of the ramp fits within the opening 454 in the push wire 450 and the blunt distal surface 648 of the ramp prevents the push wire 450 from being retracted proximally relative to the collar 640. Such a ramp-shaped protrusion 646 allows the push wire 450 to remain substantially parallel to the lumen 641 in the collar 640 during a step of coupling the push wire 450 to the collar 640. Thus, the collar 640 and push wire 450 configuration depicted in FIG. 6 does not require the push wire 450 to be rotated during the coupling step.

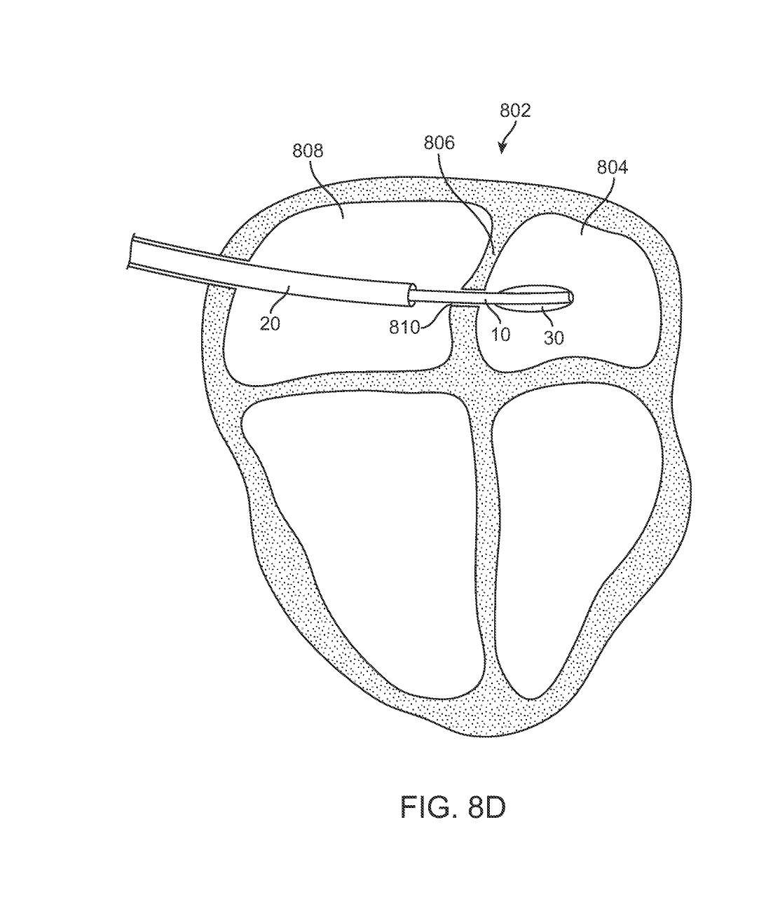

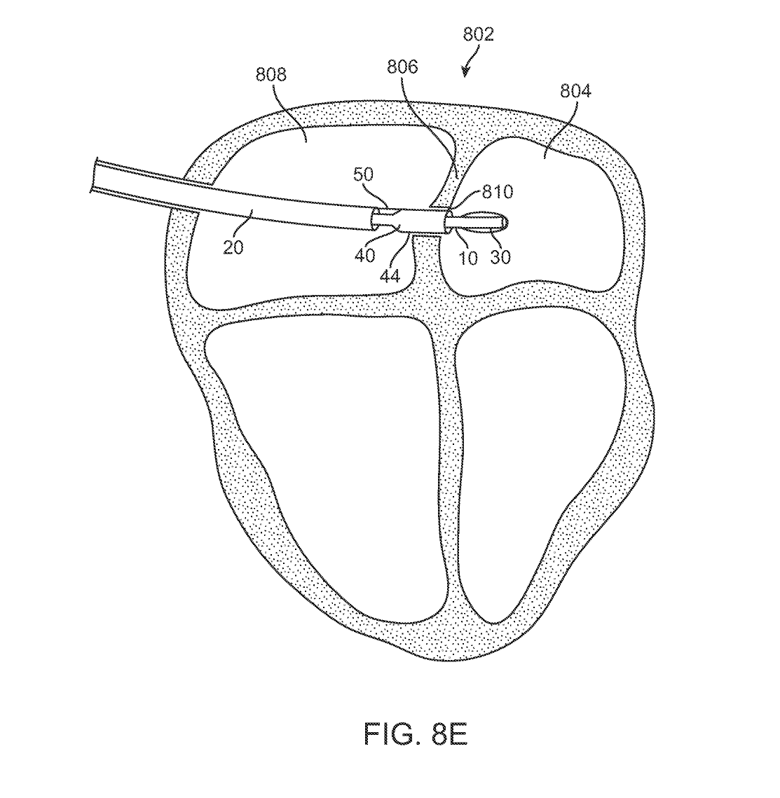

In yet another embodiment, the push wire 450 depicted in FIGS. 4A-4E can be used with a collar that includes an opening adjacent to the pin in the attachment region for receiving the distal end 452 of the push wire 450 and accommodating rotation of the push wire 450 about the pin. For example, FIG. 7A depicts a moveable collar 740 comprising an attachment region 742 with a pin 746 sized to fit within the opening 454 in the push wire 450. The opening 748 adjacent to the pin 746 is configured for receiving the distal end 452 of the push wire 450 and accommodating rotation of the push wire 450 from a perpendicular position relative to the collar lumen 741 (shown in phantom) to a parallel position relative to the collar lumen 741, as indicated by arrow 760. To this end, the opening 748 in the attachment region 742 has a semi-circular shape and retaining upper shoulder portions 749 for retaining the distal end 452 of the push wire 450 therein.

Similar to the collar 740 shown in FIG. 7A, the moveable collar 740′ shown in FIG. 7B includes an opening 748′ adjacent to the pin 746′ in the attachment region 742′ of the collar 740′. However, the opening 748′ and the pin 746′ are different shapes than the opening 748 and the pin 746 in the attachment region 742 of the collar 740. In particular, the opening 748′ has only one retaining upper shoulder portion 749′. Also similar to the embodiment shown in FIG. 7A, the opening 748′ adjacent to the pin 746′ is configured for receiving the distal end 452 of the push wire 450 and accommodating rotation of the push wire 450 from a perpendicular position relative to the collar lumen 741′ (shown in phantom) to a parallel position relative to the collar lumen 741′, as indicated by arrow 760′. To this end, the opening 748′ in the attachment region 742′ has a semi-circular shape and a retaining upper shoulder portion 749′ for retaining the distal end 452 of the push wire 450 therein.

Having described the structure of the recapturing apparatus 60, as well as means for engaging the push wire 50 to the moveable collar 40, the operation of the medical kit assembly 100 in recapturing the expandable body 30 will now be described with reference to FIGS. 8A-8F. It should be noted that, although the recapturing apparatus 60 is described as being particularly useful in recapturing the expandable body 30 and withdrawing the expandable body 30 through an atrial septum, the recapturing apparatus 60 can also be used in other methods where it is desirable to capture and/or radially compress the expandable body 30 prior to retracting the expandable body 30 into the sheath 20.

First, as shown in FIG. 8A, the guide sheath 20 is introduced into the right atrium 808 of the heart 802. A guide catheter or guide wire (not shown) may be used in association with the guide sheath 20 to aid in directing the guide sheath 20 through the appropriate artery toward the heart 802. An opening 810 (shown in FIG. 8B) is then formed within the septum 806 between the right atrium 808 and the left atrium 804. In one method, the opening 810 in the atrial septum 806 is created by puncturing the atrial septum 806 using a sharpened lead wire (not shown), as described in greater detail in U.S. Pat. No. 5,575,810, which is incorporated by reference above.

After the opening 810 is created, the catheter 10 is advanced through the sheath 20 until the balloon 30 of the catheter 10 distally deploys out from the sheath 20. The catheter 10 is further advanced from the right atrium 808 into the left atrium 804 by passing through the opening 810 in the atrial septum 806, as shown in FIG. 8B. Once the catheter 10 is properly positioned within the left atrium 804, the balloon 30 is inflated from its original geometry to an expanded geometry, as shown in FIG. 8C, and an ablation procedure is performed in a conventional manner.

After the ablation procedure is completed, the balloon 30 is deflated to a collapsed geometry, shown in FIG. 8D, which has a slightly larger profile than the original geometry shown in FIG. 8B. If the balloon 30 in the collapsed configuration has a larger profile than the opening 810 in the septum 806, it is difficult to withdraw the balloon 30 back through the opening 810 without tearing or otherwise damaging the atrial septum 806. Thus, the recapturing apparatus 60 is deployed to capture the balloon 30 and facilitate withdrawal of the balloon 30 back through the atrial septum 806.

Prior to deployment of the recapturing apparatus 60, the collar 40 is positioned at the proximal ends of the sheath 20 and the catheter 10, as shown in FIG. 1B. In order to achieve this initial configuration, in one embodiment, the catheter 10 is supplied with the moveable collar 40 disposed on the proximal end of the catheter 10. In another embodiment, prior to inserting the catheter 10 into the guide sheath 20, the moveable collar 40 is positioned within the proximal end of the guide sheath 20. In this embodiment, the distal end of the catheter 10 passes through the lumen 41 of the collar 40 and is distally advanced while retaining the collar 40 at the proximal end of the sheath 20 so that, when the balloon 30 is deployed distally out of the sheath 20, the collar 40 is disposed at the proximal end of the catheter 10. In both of these embodiments, the push wire 50 is provided separately from the moveable collar 40

Thus, after it is determined that the recapturing apparatus 60 is needed for facilitating retraction of the balloon 30, a first step in deploying the recapturing apparatus 60 is for a user to obtain the push wire 50. After obtaining the push wire 50, the push wire 50 is coupled to the attachment region 42 of the collar. Preferably, the attachment region 42 of the collar 40 protrudes proximally out of the sheath 20, as shown in FIG. 1B, so that the attachment region 42 is accessible for performing a procedure for coupling the push wire 50 to the collar 40.

The procedure for coupling the push wire 50 to the collar 40 depends on which embodiment of the recapturing apparatus 60 is used. For example, when using the recapturing apparatus shown in FIGS. 2A or 2B, the push wire 250 is coupled to the collar 240 or 240′ by interference fitting the distal end 252 of the push wire 250 into the opening 246 or 246′ in the attachment region 242 or 242′ of the collar 240 or 240′. When using the recapturing apparatus shown in FIGS. 3A-3D, the push wire 350 is coupled to the collar 340 by inserting the bent distal end 352 of the push wire 350 into the opening 346 or 346′ in the attachment region 342 of the collar 340. When using the push wire 450 in FIGS. 4A-4D, the push wire 450 is coupled to the moveable collar by receiving an attachment region pin within the opening 454 in the distal end 452 of the push wire 450, as discussed in more detail in association with FIGS. 5A-7B. When using the recapturing apparatus shown in FIGS. 5A and 5B, the push wire 450 is coupled to the collar 540 by receiving the pin 546 within the opening 454 and then rotating the push wire 450 from a perpendicular position, as shown in FIG. 5B, to a parallel position, as shown in FIG. 5A, relative to the collar lumen 541. When using the recapturing apparatus shown in FIG. 6, the push wire 450 is slid distally along the ramp-shaped protrusion 646 until the opening 454 in the push wire 450 receives the apex 649 of the ramp 646 therein. When using the recapturing apparatus shown in FIG. 7A or 7B, the push wire 450 is coupled to the collar 740 or 740′ by inserting the distal end 452 of the push wire 450 into the opening 748 or 748′ in the attachment region 742 or 742′ and then rotating the push wire 450 from a perpendicular position to a parallel position relative to the collar lumen 741 or 741′, thereby receiving the pin 746 or 746′ on the collar 740 or 740′ within the opening 454 in the push wire 450.

After the push wire 50 is coupled to the collar 40 using any of the methods described above, the collar 40 is distally advanced along the catheter 10 by distally pushing the wire 50 relative to the catheter 10. An initial stage of the distal advancement of the collar 40 is depicted in FIG. 1C. Distal advancement of the collar 40 continues until the collar 40 deploys out of the distal end of the sheath 20 and at least part of the collar 40 is positioned within the opening 810 in the atrial septum 806, as shown in FIG. 8E. Since the distal capture region 44 of the collar 40 has a larger diameter than the catheter 10, but a smaller diameter than the sheath 20, the collar 40 functions to dilate the opening 810 in the septum 806, while causing less trauma than the sheath 20 would cause.

Next, the balloon 30 is captured within the collar 40 by either distally advancing the collar 40 into the left atrium 804 or proximally retracting the catheter 10 until the balloon 30 is received within the capture region 44 of the collar 40. The collar 40 and the captured balloon 30 are then proximally retracted towards the distal end of the sheath 20, as shown in FIG. 8F. After the collar 40 and the captured balloon 30 are completely retracted into the sheath 20, the entire medical kit 100 is removed from the patient.

Although particular embodiments have been shown and described, it should be understood that the above discussion is not intended to limit the scope of these embodiments. Various changes and modifications may be made without departing from the scope of the claims. Thus, embodiments are intended to cover alternatives, modifications, and equivalents that may fall within the scope of the claims.

What is claimed is:

1. A recapturing apparatus, comprising:
a moveable collar sized to fit within a guide sheath, the collar having a distal capture region sized to fit over and radially compress an expandable body disposed at a distal end of a catheter shaft, a proximal attachment region, and a central lumen sized to receive the catheter shaft therein; and
a push wire having a distal end configured to engage the attachment region, thereby affixing the push wire to the collar.

2. The recapturing apparatus of claim 1, wherein an inner diameter of a distal end of the collar is greater than an inner diameter of a proximal end of the collar.

3. The recapturing apparatus of claim 1, wherein the distal end of the push wire is configured for being interference fit with the attachment region of the collar, and wherein the attachment region of the collar comprises an opening for receiving the distal end of the push wire therein.

4. The recapturing apparatus of claim 3, wherein the distal end of the push wire comprises flared ridges configured for engaging the opening in the attachment region of the collar, and wherein the opening in the attachment region of the collar is substantially parallel to the central lumen in the collar.

5. The recapturing apparatus of claim 3, wherein the distal end of the push wire is bent at a substantially right angle relative to a proximal portion of the push wire, and wherein the opening in the attachment region of the collar is substantially perpendicular to the central lumen in the collar.

6. The recapturing apparatus of claim 1, wherein the attachment region of the collar comprises a pin and the distal end of the push wire comprises an opening for receiving the pin therein.

7. The recapturing apparatus of claim 6, wherein the opening in the distal end of the push wire is configured to engage with the pin in the attachment region of the collar by receiving the pin within the opening and then rotating the push wire from a perpendicular position to a parallel position relative to the lumen of the collar.

8. The recapturing apparatus of claim 7, wherein the attachment region further comprises an opening adjacent to the pin, and wherein the opening in the distal end of the push wire is configured to engage with the pin in the attachment region of the collar by inserting the distal end of the push wire into the opening in the attachment region of the collar and then rotating the push wire from a perpendicular position to a parallel position relative to the lumen of the collar.

9. The recapturing apparatus of claim 7, wherein the distal end of the push wire has a quadrate cross-section and a proximal portion of the push wire has a rounded cross-section.

10. A medical system, comprising:
an elongated catheter having a distally located expandable body configured for being placed between an expanded geometry and a collapsed geometry;
a moveable collar configured for being displaced relative to the catheter to capture the expandable body therein when in the collapsed geometry;
an elongated push wire configured for engaging the moveable collar; and
an elongated sheath having a distal end, a proximal end, and a lumen extending between the proximal end and the distal end, wherein the catheter is configured for being disposed within the lumen of the sheath, and the moveable collar is configured for being disposed concentrically between the catheter and the sheath.

11. The medical system of claim 10, wherein the expandable body is a balloon.

12. The medical system of claim 10, wherein the moveable collar comprises a proximal attachment region and a distal end of the push wire is configured to engage with the attachment region of the collar, and wherein the moveable collar comprises a distal capture region sized to fit over and radially compress the expandable body when in the collapsed geometry, the system further comprising means for engaging the push wire with the moveable collar.

13. The medical system of claim 10, wherein an inner diameter of a distal end of the collar is greater than an inner diameter of a proximal end of the collar.

14. A method for performing a medical procedure using a catheter having an expandable body, comprising:
advancing the catheter through a sheath until the expandable body exits the sheath;
expanding the expandable body;
performing the medical procedure;
collapsing the expandable body after performing the medical procedure;
advancing a collar along the catheter until the expandable body is captured within the collar; and
retracting the collar into the sheath.

15. The method of claim 14, further comprising moving the expandable body from a first anatomical cavity into a second anatomical cavity by passing through a septum between the respective cavities, wherein expanding the expandable body, performing the medical procedure, collapsing the expandable body, and advancing the collar are performed while the expandable body is in the second cavity.

16. The method of claim 15, further comprising withdrawing the collar and the captured expandable body from the second cavity into the first cavity by passing through the septum prior to retracting the collar into the sheath.

17. The method of claim 15, wherein the expandable body is passed through an opening within the septum, and wherein the expandable body, after the step of collapsing the expandable body, has a greater profile than the opening.

18. The method of claim 15, wherein a distal end of the sheath remains in the first cavity while the expandable body is moved from the first cavity into the second cavity.

19. The method of claim 14, further comprising coupling an elongated push wire to the collar, wherein the collar is advanced along the catheter by advancing the push wire relative to the catheter, and wherein coupling the push wire to the collar comprises interference fitting the push wire to the collar.

20. The method of claim 14, wherein retracting the collar into the sheath further comprises retracting the captured expandable body into the sheath.
